(19) United States
(12) Plant Patent Application Publication
(10) Pub. No.: US 2019/0183003 P1
(43) Pub. Date: Jun. 13, 2019

(54) Cannabis plant named 'LEMON CRUSH'

(50) ;

(71) Applicant: Biotech Institute LLC, Westlake Village, CA (US)

(72) Inventors: Mark A. Lewis, Westlake Village, CA (US); Steven Haba, Westlake Village, CA (US)

(21) Appl. No.: 15/932,562

(22) Filed: Mar. 16, 2018

Related U.S. Application Data
(60) Provisional application No. 62/596,561, filed on Dec. 8, 2017.

(51) Int. Cl. A01H 6/28

(52) U.S. Cl. PLT/263.1

(57) ABSTRACT

The present invention provides a new and distinct cannabis cultivar designated as ‘LEMON CRUSH’.

### CROSS-REFERENCE TO RELATED APPLICATIONS

This application claims priority to, and the benefit of U.S. Provisional Patent Application Ser. No. 62/596,561, filed Dec. 8, 2017, which is hereby incorporated by reference in its entirety for all purposes.

Latin name of genus and species: Cannabis hybrid (mixed background).

Variety denomination: ‘LEMON CRUSH’.

### BACKGROUND OF THE INVENTION

The present invention relates to a new and distinct cannabis cultivar designated as ‘LEMON CRUSH’.

This new cultivar is the result of controlled-crosses between proprietary cultivars made by the inventors. The new cultivar of ‘LEMON CRUSH’ was asexually reproduced via a ‘cutting’ and ‘cloning’ method by the inventors at Salinas, Calif. Asexual clones from the original source have been tested in greenhouses, nurseries, and/or fields. The properties of each cultivar were found to be transmissible by such asexual reproduction. The cultivar is stable and reproduces true to type in successive generations of asexual reproduction.

### TAXONOMY AND NOMENCLATURE

Cannabis, more commonly known as marijuana, is a genus of flowering plants that includes at least three species, Cannabis sativa, Cannabis indica, and Cannabis ruderalis as determined by plant phenotypes and secondary metabolite profiles. In practice however, cannabis nomenclature is often used incorrectly or interchangeably. Cannabis literature can be found referring to all cannabis varieties as “sativas” or all cannabinoid producing plants as “indicas”. Indeed the promiscuous crosses of indoor cannabis breeding programs have made it difficult to distinguish varieties, with most cannabis being sold in the United States having features of both sativa and indica species.

Human cultivation history of Cannabis dates back 8000 years. Schultes, R. E. 1970. Random thoughts and queries on the botany of Cannabis. Pages 11-38 in: C R B Joyce, and S H Curry eds., THE BOTANY AND CHEMISTRY OF CANNABIS. J. & A. Churchill. London, England. Hemp cloth recovered in Europe dates back 6000 years. (Small, E, Beckstead, H D, and Chan, A, 29(3) ECONOMIC BOTANY 29(3): 219-232 (1975). The written record of the pharmacologic properties of Cannabis goes back more than 4000 years. Ti, H. 2737 BC. NEI JING SU WEN HUANG TI (Yellow Emperor's Classic on Internal Medicine; referred to without citation in Small et al. 1975 Supra).

The taxonomy and nomenclature of the highly variable genus Cannabis (Emboden, W A, 29(3) ECONOMIC BOTANY 304-310 (1974)); (Small, E and Cronquist, A, 25(4) TAXON 405-435 (1976)); Small E and Cronquist, A 26(1) TAXON 110 (1977)); (Hillig, K W and Mahlberg, P G, 91(6) American Journal of Botany 966-975 (2004)), remains in question. This is in spite of the fact that its formal scientific name, ‘Cannabis sativa L.’, assigned by Carolus Linneaus (Linnaeus, C, 2 SPECIES PLANTARUM 1027 (1753), Salvius, Stockholm. Facsimile edition, 1957-1959. Ray Society, London, U.K.), is one of the oldest established names in botanical history and is still accepted to this day. Another species in the genus, ‘Cannabis indica Lam.’ was formally named somewhat later (de Lamarck, J B, 1(2) ENCYCLOPEDIE METHODIQUE DE BOTANIQUE, 694-5,(1785)), but is still very old in botanical history. In 1785, Jean-Baptiste Lamarck published a description of a second species of Cannabis, which he named Cannabis indica. Lamarck based his description of the newly named species on plant specimens collected in India. C. indica was described as relatively short, conical, and densely branched, whereas C. sativa was described as tall and laxly branched (Schultes R. E. et al Harvard University Botanical Museum Leaflets, 23: 337-367 (1974)). C. indica plants was also described as having short, broad leaflets whereas those of C. sativa were characterized as relatively long and narrow (Anderson L. C., Harvard University Botanical Museum Leaflets, 28: 61-69 (1980)). C. indica plants conforming to Schultes's and Anderson's descriptions may have originated from the Hindu Kush mountain range. Because of the often harsh and variable (extremely cold winters, and warm summers) climate of those parts, C. indica is well-suited for cultivation in temperate climates.

Three other species names were proposed in the 1800s to distinguish plants with presumably different characteristics (C. macrosperma Stokes, C. chinensis Delile, C. gigantean Vilmorin), none of which are accepted today, although the epithet “indica” lives on as a subspecies of C. sativa (‘C. sativa ssp. indica Lam.’. Small and Cronquist 1976 Supra).

In the 20th century, two new names were added to the liturgy of proposed ‘Cannabis species: C. ruderalis’ Janischevsky and a hybrid, x ‘C. intersita’ Sojak. Small, E, Jui, P Y, and Lefkovitch, L P, 1(1) SYSTEMATIC BOTANY 1(1): 67-84 (1976); Small and Cronquist 1976, Supra. Further, numerous names have been proposed for horticultural variants of ‘Cannabis’ but as of 1976, “very few of these have been validly published as formal taxa under the International Code of Botanical Nomenclature.” Small and Cronquist 1976 Supra. Moreover, other recent work continues to focus on higher-order evolutionary relationships of the genus. Cannabis has been variously ascribed as belonging to the mulberry family (Moraceae) (Engler, H G A, Ulmaceae, Moraceae and Urticaceae, pages 59-118 (1889) in: A. Engler and K. Prantl eds., DIE NATURLICHEN PFLANZENFAMILIEN 3(1). W. Engelmann, Leipzig, Germany; Judd, W S, Sanders, R W, and Donoghue, M J, 5 HARVARD PAPERS IN BOTANY 5: 1-51 (1994)); nettle family (Urticaceae) (Berg, C C, Systematics and phylogeny of the Urticales, pages 193-220, in: P. R. Crane and S. Blackmore eds., EVOLUTION, SYSTEMATIC, AND FOSSIL HISTORY OF THE HAMAMELIDAE, VOL. 2, HIGHER HAMAMELIDAE, Clarendon Press, Oxford, U.K. (1989); Humpries, C J and Blackmore, S, A review of the classification of the Morcaceae, pages 267-277 In: Crane and Blackmore 1989 id.); and most recently in its own family with hops (Humulus), the Cannabaceae, or hemp family. Sytsma, K J, et al, 89(9) AMERICAN JOURNAL OF BOTANY 1531-1546 (2002). While the work of Small and Cronquist 1976 Supra, seemed to effectively confine the genus to a single species with 2 subspecies (C. sativa s., C. s. indica), each with two varieties (C. s. s. var. sativa, C. s. s. var. spontanea; C. s. i. var. indica, C. s. i. var. Kafiristanica) largely on the basis of chemotaxonomy and interfertility of all forms, more recent work (Systma et al. 2002, Supra), proposes a two-species concept, resurrecting the binomial C. indica Lam. Since Sytsma et al. 2002 provides no key for discriminating between the species, the dichotomous key of Small and Cronquist 1976 Supra, which accounts for all forms in nature, whether wild or domesticated, is preferred to classify the characteristics of the plants.

### BRIEF SUMMARY OF THE INVENTION

This invention relates to a new and distinctive cannabis cultivar designated as ‘LEMON CRUSH’.

The objective of the breeding program which produced novel plants disclosed herein was primarily to develop a cannabis cultivar with its unique blend of various cannabinoids and/or terpenes for (a) medicinal effects such as improving appetite and reducing nausea, vomiting and/or chronic pain, as well as neurological and cardiovascular effects, and (b) psychoactive effects such as increased motivation and energetic behavior rather than indifference, passiveness and lethargy, and (c) recreational effects with enhanced enjoyment such as food and aroma.

As used herein, the term “cultivar” is used interchangeably with “variety”, “strain”, and/or “clone”.

Cannabis plants produce a unique family of terpeno-phenolic compounds called cannabinoids. Cannabinoids, terpenoids, and other compounds are secreted by glandular trichomes that occur most abundantly on the floral calyxes and bracts of female plants. As a drug it usually comes in the form of dried flower buds (marijuana), resin (hashish), or various extracts collectively known as hashish oil. The cannabis plant has at least 545 distinct compounds that span 20 chemical classes including cannabinoids, terpenes, terpenoids, amino acids, nitrogenous compounds, simple alcohols, aldehydes, ketones, esters, lactones, acids, fatty acids, steroids, non-cannabinoid phenols, pigments, flavonoids, vitamins, proteins, enzymes, glycoproteins, and hydrocarbons. Terpenes and/or cannabinoids, in particular, have shown great potential in terms of medicinal value.

Terpenes and/or cannabinoids have been shown to be largely responsible for beneficial effects of a cannabis pant. In fact, each cannabis plant has the varying concentrations of medically viable compounds depending on different strains (genotypes) and their resulting chemotypes. Even a small variation in terpene and/or cannabinoid concentration can cause noticeable differences in the entourage and/or synergistic effects of a cannabis plant, which distinguishes one variety from another. Research shows that it relies heavily on the physiological effects produced by terpenes and/or cannabinoids.

Over 100 different kinds of terpenes have been identified in cannabis plants although not being as well-studied as cannabinoids they are instrumental in giving rise to the physiological and psychoactive effects in cannabis.

Terpenes are a large and diverse class of organic compounds, produced by a variety of plants. They are often strong smelling and thus may have had a protective function. Terpenes are an important component, not only influencing taste and smell of each cannabis strain but also influencing its effects on the mind and body of a subject such as humans and animals. Terpenes are a classification of organic molecules that are found in a wide variety of plants and animals. These molecules are known for their characteristic scents and flavors. The varying terpene concentrations found in cannabis plants directly influence the resulting taste and smell, as well as the observed effects. Non-limiting examples of terpenes include Hemiterpenes, Monoterpenes, Sesquiterpenes, Diterpenes, Sesterterpenes, Triterpenes, Sesquarterpenes, Tetraterpenes, Polyterpenes, and Norisoprenoids. The main terpenes found in cannabis plants are myrcene, limonene, caryophyllene, pinene, terpinene, terpinolene, camphene, terpineol, phellandrene, carene, humulene, pulegone, sabinene, geraniol, linalool, fenchol, berneol, eucalyptol, and nerolidon.

Cannabinoids are the most studied group of the main physiologically active secondary metabolites in cannabis. The classical cannabinoids are concentrated in a viscous resin produced in structures known as glandular trichomes. At least 113 different cannabinoids have been isolated from cannabis plants. The main classes of cannabinoids from cannabis include tetrahydrocannabinol (THC), cannabidiol (CBD), cannabigerol (CBG), and cannabinol (CBN). Cannabinoid can be at least one of a group comprising tetrahydrocannabinol (THC), cannabidiol (CBD), cannabigerol (CBG), cannabinol (CBN) cannabichromene (CBC), cannabinodiol (CBDL), cannabicyclol (CBL), cannabivarin (CBV), tetrahydrocannabivarin (THCV), cannabidivarin (CBDV), cannabigerovarin (CBGV), cannabichromevarin (CBCV), cannabigerol monomethyl ether (CBGM), cannabielsoin (CBE), cannabicitran (CBT), cannabinol propyl variant (CBNV), cannabitriol (CBO), tetrahydrocannabinolic acid (THCA), tetrahydrocannabivarinic acid (THCVA), cannabidiolic acid (CBDA), cannabigerolic acid (CBGA) and cannabinerolic acid.

Most cannabinoids exist in two forms, as acids and in neutral (decarboxylated) forms. The acid form is designated by an “A” at the end of its acronym (i.e. THCA). The cannabinoids in their acid forms (those ending in “-A”) can be converted to their non-acid forms through a process called decarboxylation when the sample is heated. The phytocannabinoids are synthesized in the plant as acid forms, and while some decarboxylation does occur in the plant, it increases significantly post-harvest and the kinetics increase at high temperatures (Flores-Sanchez and Verpoorte, Plant Cell Physiol. 49(12): 1767-1782 (2008)). The biologically active forms for human consumption are the neutral forms. Decarboxylation is usually achieved by thorough drying of the plant material followed by heating it, often by either combustion, vaporization, or heating or baking in an oven. Unless otherwise noted, references to cannabinoids in a plant include both the acidic and decarboxylated versions (e.g., CBD and CBDA).

The molecules lose mass through the process of decarboxylation. In order to find the total theoretical active cannabinoids, the acid forms should be multiplied by 87.7%. For example, THCA can be converted to active THC using the formula: THCA×0.877=THC. The maximum THC for the sample is: THCₘₐₓ=(THCA×0.877)+THC. This method has been validated according to the principles of the International Conference on Harmonization. Similarly, CBDA can be converted to active CBD and the yield is determined using the yield formula: CBDA×0.877=CBD. Also the maximum amount of CBD yielded, i.e max CBD for the sample is: CBDₘₐₓ=(CBDA×0.877)+CBD. Additionally, CBGA can be converted to active CBG by multiplying 87.8% to CBGA. Thus, the maximum amount of CBG is: CBGₘₐₓ=(CBGA×0.878)+CBG.

The biologically active chemicals found in plants, phytochemicals, affect the normal structure or function of the human body and in some cases treat disease. The mechanisms for the medicinal and psychoactive properties of a cannabis plant, like any medicinal herb, produce the pharmacologic effects of its phytochemicals, and the key phytochemicals for a medical cannabis plant are cannabinoids and terpenes.

While Δ9-Tetrahydrocannabinol (THC) is also implicated in the treatment of disease, the psychotropic activity of THC makes it undesirable for some patients and/or indications.

Tetrahydrocannabinol, THC, is the primary psychoactive and medicinal cannabinoid and is the result of the decarboxylation of tetrahydrocannabinolic acid (THC-A), its acidic precursor. THC-A, (6ar,10ar)-1-hydroxy-6,6,9-trimethyl-3-pentyl-6a,7,8,10a-tetrahydro-6h-benzochromene-2-carboxylic acid, is found in the trichomes of the plant and converted into THC, which actually exists in only minute quantities in the living plant, after harvest and drying.

While Cannabigerol (CBG), is not considered psychoactive, it is known to block the psychoactive effects of THC and is considered medically active in a variety of conditions. Its precursor, cannabigerolic acid, CBG-A, (E)-3-(3,7-Dimethyl-2,6-octadienyl)-2,4-dihydroxy-6-pentylbenzoic acid, is being studied medically.

Delta-9-Tetrahydrocannabinol or (THC) is a psychoactive cannabinoid responsible for many of the effects such as mild to moderate pain relief, relaxation, insomnia and appetite stimulation. THC has been demonstrated to have anti-depressant effects. The majority of strains range from 12-21% THC with very potent and carefully prepared strains reaching even higher.

Cannabidiol (CBD) is one of the principal cannabinoids found in a cannabis plant and is largely considered to the most medically significant. CBD occurs in many strains, at low levels, <1%. In rare cases, CBD can be the dominant cannabinoid, as high as 15% by weight. CBD is non-psychoactive, meaning that unlike THC, CBD does not cause a noticeable “high”. CBD has been implicated for medical properties in the treatment of a wide variety of diseases and symptoms, including cancer, nausea, chronic pain, spasms, seizures/epilepsy, anxiety, psoriasis, Crohn's disease, rheumatoid arthritis, diabetes, schizophrenia, post-traumatic stress disorder (PTSD), alcoholism, strokes, Multiple Sclerosis, and cardiovascular disease. CBD also has been reported to act as a muscle relaxant, antibiotic, anti-inflammatory, and bone stimulant, as well as to improve blood circulation, cause drowsiness, and protect the nervous system. It can provide relief for chronic pain due to muscle spasticity, convulsions and inflammation, as well as effective relief from anxiety-related disorders. Offering relief for patients with Multiple Sclerosis (MS), Fibromyalgia and Epilepsy. CBD has also been shown to inhibit cancer cell growth when injected into breast and brain tumors in combination with THC.

A cannabis cultivar can be used to achieve the desire of patients to be treated with CBD without the adverse side-effects (e.g., psychoactivity) of THC.

Cannabichromene (CBC) is a rare, non-psychoactive cannabinoid, usually found at low levels (<1%) when present. It has been shown to have anti-depressant effects and to improve the pain-relieving effects of THC. Studies have demonstrated that CBC has sedative effects such as promoting relaxation.

Cannabigerol (CBG) is a non-psychoactive cannabinoid. CBG-acid is the precursor to both THC-acid and CBD-acid in the plant usually found at low levels (<1%) when present. It has been demonstrated to have both pain relieving and inflammation reducing effects. CBG reduces intraocular pressure, associated with glaucoma. CBG has been shown to have antibiotic properties and to inhibit platelet aggregation, which slows the rate of blood clotting.

Cannabidiol (CBD) and cannabichromene (CBC) are both non-psychoactive and end products of CBG metabolism, like THC, that are used medically Cannabichromenic acid.

Cannabinol or (CBN) is an oxidative degradation product of THC. It may result from improper storage or curing and extensive processing, such as when making concentrates. It is usually formed when THC is exposed to UV light and oxygen over time. CBN has some psychoactive properties, less strength than THC.

High potency cannabis plants contain large quantities of specific terpenes as well as various assortments of others. For instance, a cannabis plant may have a profile with a high level of, a moderate amount of and a small amount of various terpenes depending on its cultivar and environmental conditons.

Various cultivars of ‘Cannabis’ species have been cultivated in an effort to create a cultivar best suited to meet the interest of inventor(s) according to its own need. The particular plant disclosed herein was discovered in the area where the inventor was intentionally cross-pollinating and cultivating plants described below using standard Mendelian breeding procedures well known to those of ordinary skill in the art. This resulted in the progenies of the inventors' crosses.

The progeny resulting from any selection stage of either the crossing, selfing or backcrossing versions of the breeding regimes of the present invention were asexually reproduced to fix and maintain the desirable THC content, CBs content, terpenes content, the aroma and flavor(s) typical of the desired class, and the other desirable phenotypic and/or genotypic characteristics. The resultant selected cannabis cultivar is designated as ‘LEMON CRUSH’ disclosed herein.

The inventor reproduced progenies asexually by cutting and cloning. This is the origin of this remarkable new cultivar. The plant has been and continues to be asexually reproduced by cutting and cloning at the inventor's greenhouses, nurseries and/or fields in Salinas, Calif., Oakland, Calif., and/or Washington, D.C.

The following are the most outstanding and distinguishing chemical characteristics of this new cultivar when grown under normal conditions in Salinas, Calif. Chemical analyses of the parental and progeny cannabis varieties disclosed herein were performed using standard chemical separation techniques well known to those skilled in the art. Samples for assaying were obtained from flower tissues of the cannabis plant disclosed herein. Cannabinoid composition of this cultivar can be determined by assaying the concentration of at least one cannabinoid in a subset (e.g., sample) of the harvested product.

Table 1 includes detailed information of the cannabis plant named ‘LEMON CRUSH’ for concentration ranges of terpenes and cannabinoids as tested on flowers sampled on at least four different dates. The cannabis plant has been tested in a laboratory setting and/or facility to determine cannabinoids and terpenes concentrations in the cannabis plant named ‘LEMON CRUSH’.
1) The main terpenes found in ‘LEMON CRUSH’ are limonene, beta-caryophyllene, alpha-humulene, linalool, trans-ocimene, beta-pinene, fenchol, alpha-terpineol, alpha-pinene and myrcene;
2) The estimated concentration of the total THCₘₐₓ, CBDₘₐₓ, and CBGₘₐₓ is about 18.77˜23.19%, about 0%, and about 0.98˜1.78%, respectively, at the time of assaying metabolites from flower samples of ‘LEMON CRUSH’; and
3) Harvest interval, i.e at 56-70 days under short day conditions.

Terpene and cannabinoid profiles of ‘LEMON CRUSH’ demonstrate that ‘LEMON CRUSH’ has a phenotypically unique profile, particular insofar as to the level of terpenes and cannabinoids. This data is presented in tabular form in Table 1

TABLE 1
Ranges of Active Cannabinoids (% by weight)

| | | | | | |
|---|---|---|---|---|---|
| Max THC | 18.77~23.19% | Max CBD | 0.00% | Max CBG | 0.98~1.78% |

Terpenes (% by weight)

| | | | | | |
|---|---|---|---|---|---|
| thujene | 0.00% | trans-ocimene | 0.15~0.33% | hexyl hexanoate | 0.04~0.08% |
| alpha-pinene | 0.09~0.17% | gamma-terpinene | 0.00% | octyl butyrate | 0.00% |
| camphene | 0.02~0.03% | linalool oxide | 0.00~0.01% | beta-caryophyllene | 0.52~0.89% |
| sabinene | 0.00% | terpinolene | 0.01~0.02% | alpha-humulene | 0.31~0.50% |
| beta-pinene | 0.14~0.20% | linalool | 0.20~0.44% | cis-nerolidol | 0.00~0.02% |
| myrcene | 0.07~0.13% | fenchol | 0.09~0.16% | trans-nerolidol | 0.00~0.04% |
| alpha-phellandrene | 0.00% | MT_1124 | 0.06~0.11% | caryophyllene oxide | 0.01~0.02% |
| carene | 0.00% | isoborneol | 0.00~0.02% | alpha-bisabolol | 0.00~0.01% |
| alpha-terpinene | 0.00% | (−) borneol | 0.02~0.04% | nerol | 0.00% |
| limonene | 0.81~1.26% | hexyl butyrate | 0.00% | geraniol | 0.00% |
| beta-phellandrene | 0.00% | alpha-terpineol | 0.08~0.15% | geranyl-acetate | 0.00~0.02% |
| cineole | 0.00~0.01% | hexyl hexanoate | N/A | methyl-eugenol | 0.00~0.02% |
| cis-ocimene | 0.00~0.01% | citronellol | 0.00~0.01% | Total Terpenes | 3.23~4.07% |

The cannabis plant named ‘LEMON CRUSH’ has a complement of terpenes, including but not limited to, relatively high levels of limonene, beta-caryophyllene, alpha-humulene, linalool, trans-ocimene, beta-pinene, fenchol, alpha-terpineol, alpha-pinene and myrcene compared to other terpene compounds. This unique combination of differently concentrated terpenes further distinguishes ‘LEMON CRUSH’ from other varieties in its odor, its medical qualities, and its effects on mood and mentation.

Physically, there are indications that its use may prevent some cancers and may prevent and/or treat diseases. Indications are that ‘LEMON CRUSH’ has many medical qualities that make it an important tool to maintain health and deal with illness.

### Asexual Reproduction

Asexual reproduction, also known as “cloning”, is a process well known to those of ordinary skill in the art of cannabis production and breeding and includes the following steps.

The cannabis cultivar disclosed herein is asexually propagated via taking cuttings of shoots and putting them in rock wool cubes. These cubes are presoaked with pH adjusted water and kept warm (˜80° C.). Full trays are covered, left under 18 hours of light and allowed to root (7-14 days). Upon root onset, the plantlets are transplanted into rigid 1 gallon containers filled with a proprietary soil mix A and remain in 18 hours of daylight for another 14-21 days. Once root-bound, plants are transplanted into rigid 3 gallon containers filled with proprietary soil mix B. Immediately, the light cycle is altered to 12/12 and flower initiating begins. The plants remain in 12/12 lighting until harvesting. They undergo a propriety nutrient regimen and grow as undisturbed as possible for 60-70 days depending on chemotype analysis.

All sun leaves are removed and plant is dismantled to result in approximately 12″ branches covered in inflorescences and trichomes. The goal in harvesting is to actually harvest trichome heads but not ‘buds’. Thus, great care is taken not to disturb the trichome heads and as much of the plant remains intact as possible to promote even and slow drying. Slow drying is followed by a one to two months curing process.

Observation of the all female progenies of the original plant has demonstrated that this new and distinct cultivar has fulfilled the objectives and that its distinctive characteristics are firmly fixed and hold true from generation to generation vegetatively propagated from the original plant.

Under careful observation, the unique characteristics of the new cultivar have been uniform, stable and reproduced true to type in successive generations of asexual reproduction.

### DESCRIPTION OF THE DRAWINGS

The accompanying color photographs depict characteristics of the new ‘LEMON CRUSH’ plants as nearly true as possible to make color reproductions. The overall appearance of the ‘LEMON CRUSH’ plants in the photographs is shown in the colors that may differ slightly from the color values described in the detailed botanical description.

FIG. 1 shows an overall view of the ‘LEMON CRUSH’ plant from the side.

FIG. 2A shows an overall view of the parental cultivar BLK03 (pollen acceptor) from above. FIG. 2B shows an overall view of the parental cultivar SLV09 (pollen donor) from above. FIG. 2C shows an overall view of the ‘LEMON CRUSH’ plant from above.

FIG. 3A shows top parts (including inflorescence) of the parental cultivar BLK03 (pollen acceptor) from the side. FIG. 3B shows top parts (including inflorescence) of the parental cultivar SLV09 (pollen donor) from the side. FIG. 3C shows top parts (including inflorescence) of the ‘LEMON CRUSH ’ plant from the side.

FIGS. 4A and 4B show a close view of flowers of the ‘LEMON CRUSH’ plant at premature and/or early floral stage.

FIGS. 5A and 5B show a close view of flowers of the ‘LEMON CRUSH’ plant at the early and/or peak floral stage.

FIGS. 6A and 6B show a close view of flowers of the ‘LEMON CRUSH’ plant at the late floral and/or senescence stage.

FIG. 7 shows another close view of flowers of the ‘LEMON CRUSH’ plant at the late floral and/or senescence stage

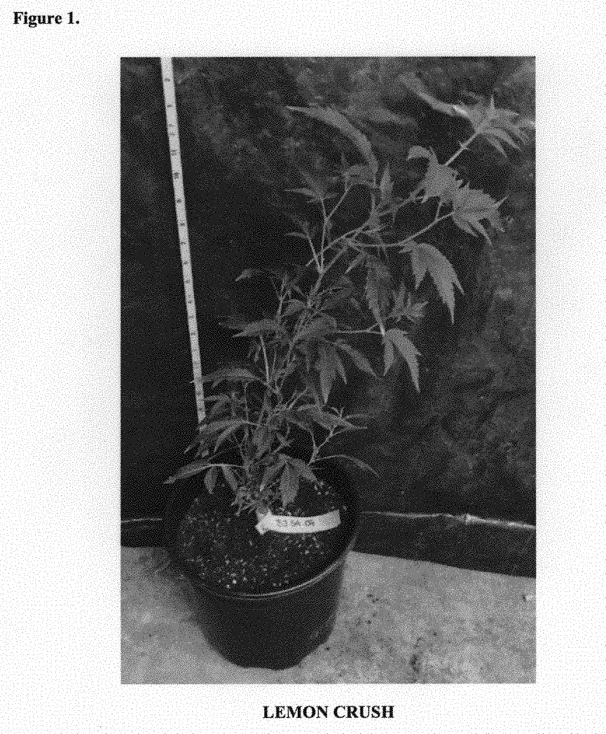

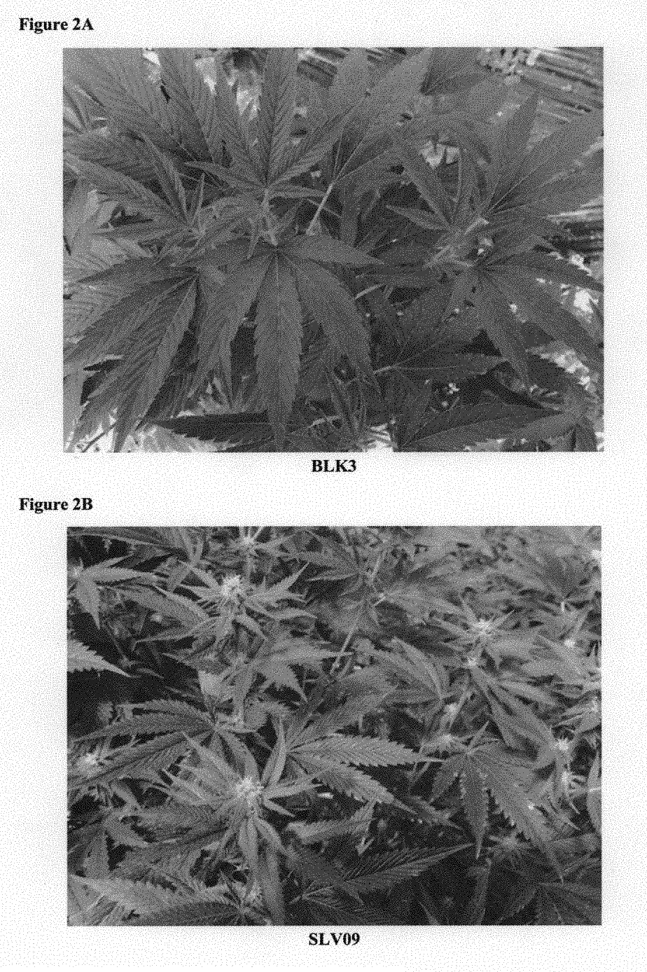

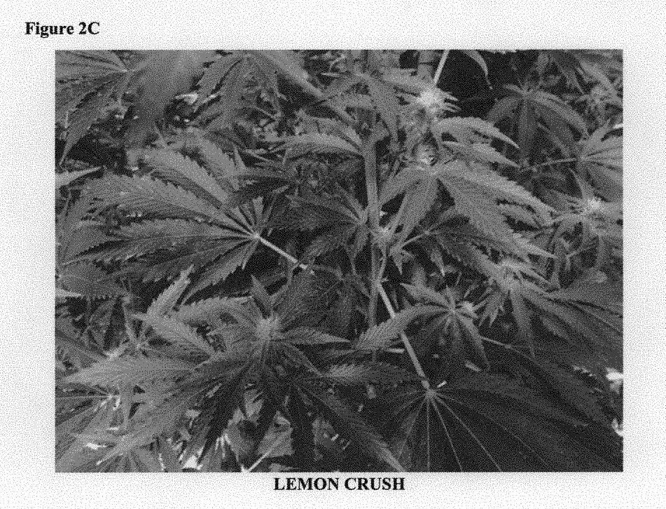

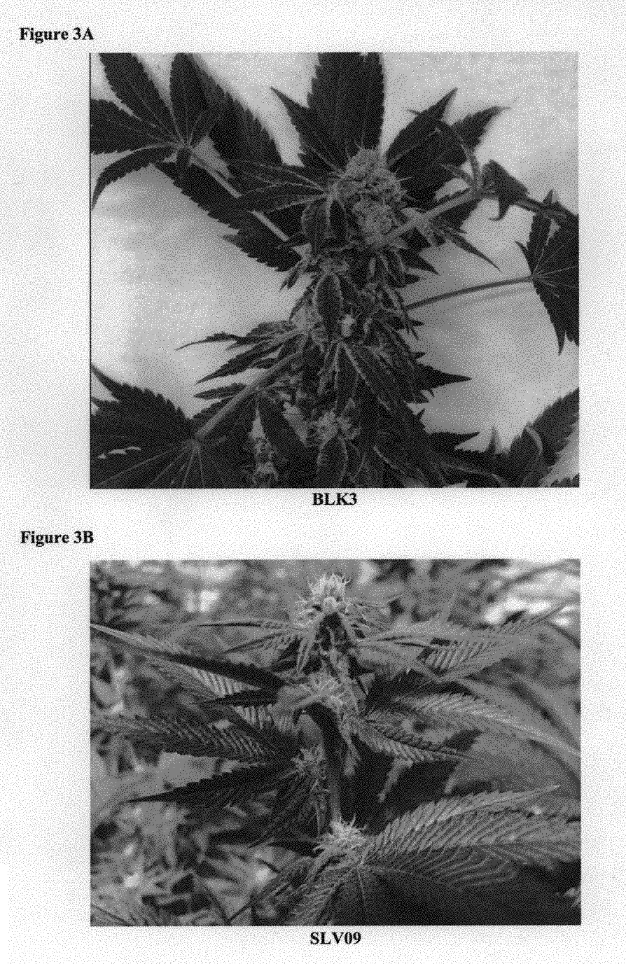

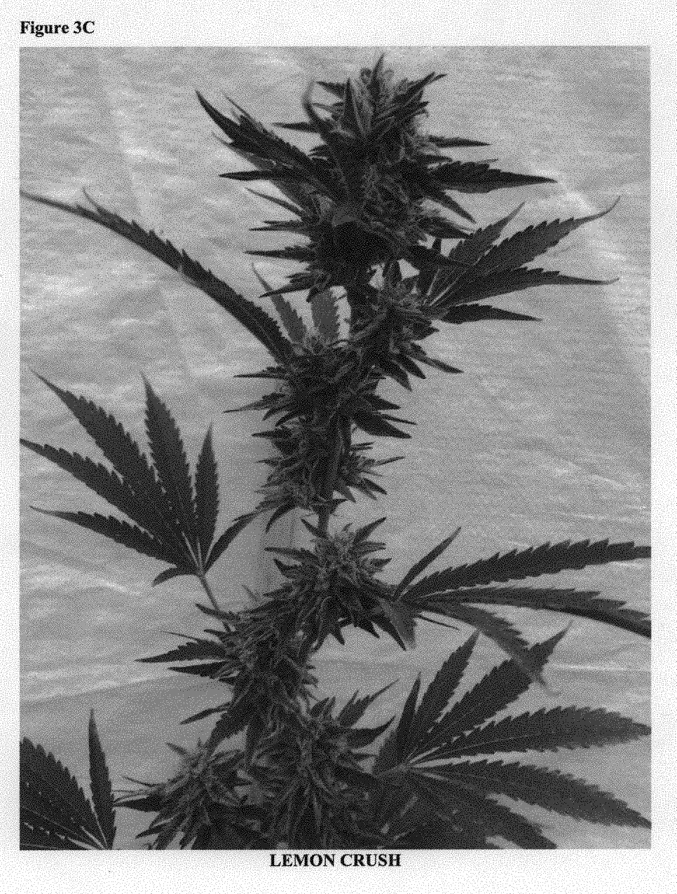

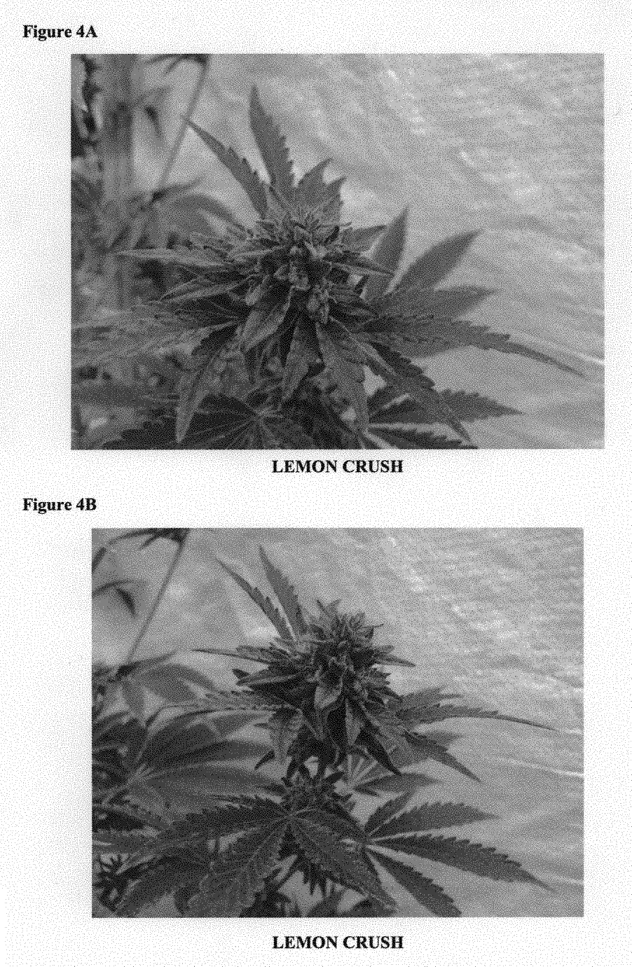

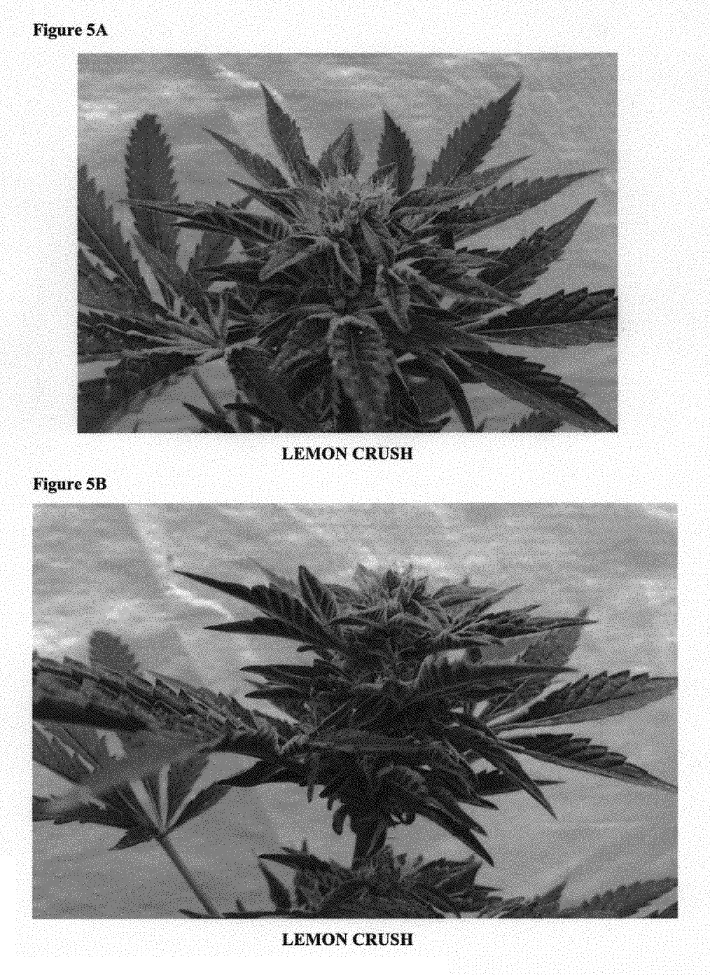

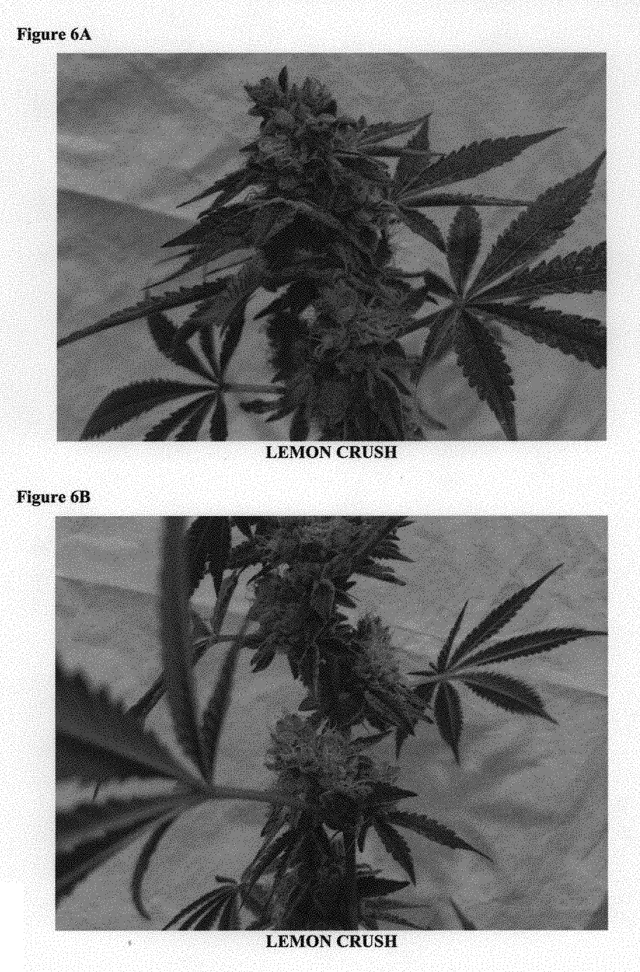

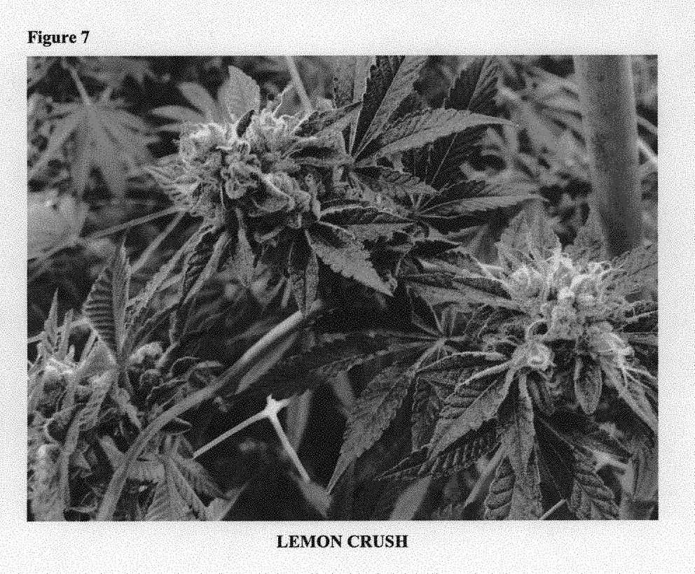

### DETAILED BOTANICAL DESCRIPTION

‘LEMON CRUSH’ has not been observed under all possible environmental conditions, and the phenotype may vary significantly with variations in environment. The following observations, measurements, and comparisons describe this plant as grown at Salinas, Calif., when grown in the greenhouse, nursery or field, unless otherwise noted.

Plants for the botanical measurements in the present application are annual plants. In the following description, the color determination is in accordance with The Royal Horticultural Society Colour Chart, 1995 Edition, except where general color terms of ordinary dictionary significance are used.

The cannabis plant disclosed herein was derived from female and male parents that are said to have been internally designated as below.

A GNBR internal Code of the cannabis plant named ‘LEMON CRUSH’ is B3.S9.09. The variety name of ‘LEMON CRUSH’ is BLK03.SLV09.09. ‘LEMON CRUSH’ is a fertile hybrid derived from a controlled-cross between two proprietary cultivars BLK03 (pollen accepter; female parent) and SLV09 (pollen donor; male parent). A GNBR Breeding Code is (B03)x(S09).09. The initial cross between two parental cultivars was made in May 2015. The phenotypic criteria to select a new and distinct cannabis cultivar disclosed herein is as follows: structure score, nose/organoleptic, mold susceptibility/resistance, and insect susceptibility/resistance. Also, the first asexual propagation of ‘LEMON CRUSH’ occurred on Sep. 26, 2016 in Salinas, Calif.

The following traits in combination further distinguish the cannabis cultivar ‘LEMON CRUSH’ from check varieties, which are the female and male parents of the cannabis cultivar disclosed and claimed herein. ‘LEMON CRUSH’ has been compared to, and can be distinguished from the parental check varieties, ‘BLK03’ and ‘SLV09’, as follows. All plants were raised together and evaluated when 93-100 days old (i.e., the day range for propagation, vegetative, and flowering times).

### I. General

| | | Parental variety(B3) | Parental variety(S9) |
|---|---|---|---|
| Characteristics | New Variety | (Female plant) | (Male plant) |
| Plant life forms | An herbaceous plant | An herbaceous plant | An herbaceous plant |
| | (herb) | (herb) | (herb) |
| Plant growth | An upright, tap-rooted | An upright, tap-rooted | An upright, tap-rooted |
| habit | annual plant | annual plant | annual plant |
| Plant origin | BLK03 × SLV09 | GLD13 × BSIA | (NL#5 × SB Purps) × |
| | | | (GlD13) |
| Plant | Asexually propagated | Asexually propagated | Asexually propagated |
| propagation | by cuttings and cloning | by cuttings and cloning | by cuttings and cloning |
| Propagation ease | Easy | Moderate | Moderate |
| Height | l.5-4 m | 0.5-2.5 m | 2.0-3.5 m |
| Width | 89 cm | 119.5 cm | 56 cm |
| Plant vigor | High | Medium | Medium |
| Time to Harvest | 11 weeks | 8 weeks | 11 weeks |
| Resistance to | Resistant | Resistant | Resistant |
| pests or diseases | | | |
| Genetically- | NO | NO | NO |
| modified | | | |
| organism | | | |

### II. Leaf/Foliage

| | | Parental variety(B3) | Parental variety(S9) |
|---|---|---|---|
| Characteristics | New Variety | (Female plant) | (Male plant) |
| Leaf | Alternate | Alternate | Alternate |
| arrangement | | | |
| Leaf shape | Palmately compound | Palmately compound | Palmately compound |
| Leaf structure | Linear-lanceolate leaflet | Linear-lanceolate leaflet | Linear-lanceolate leaflet |
| | blades with glandular | blades with glandular | blades with glandular |
| | hairs | hairs | hairs |
| Leaf margins | Dentate, coarsely | Dentate, coarsely | Dentate, coarsely |
| | serrated, and the teeth | serrated, and the teeth | serrated, and the teeth |
| | point towards the tip | point towards the tip | point towards the tip |
| Leaf hairs | Present | Present | Present |
| Leaf length | 19.1 cm | 16.6 cm | 9.50 cm |
| with petiole at | | | |
| maturity | | | |
| Petiole length | 5.50 cm | 6.50 cm | 2.00 cm |
| at maturity | | | |
| Stipule length | 0.50 cm | 0.70 cm | 0.4 cm |
| at maturity | | | |
| Stipule shape | Acute-bulbous | Elliptical | Scale-like-linear |
| No. of leaflets | 3-9 | 5-7 | 3-5 |
| Middle largest | 13.4 cm | 9.80 cm | 7.60 cm |
| (longest) leaflet | | | |
| length | | | |
| Middle largest | 2.60 cm | 2.30 cm | 1.80 cm |
| (longest) leaflet | | | |
| width | | | |
| Middle largest | 13.4:2.6 | 9.8:2.3 | 7.6:1.8 |
| (longest) leaflet | | | |
| length/width | | | |
| ratio | | | |
| No. teeth of | 29 | 25 | 23 |
| middle leaflet | | | |
| (average) | | | |
| Leaf (upper | 139a | 132a | 135b |
| side) color | | | |
| (RMS No.) | | | |
| Leaf (lower | 139c | 134d | 135b |
| side) color | | | |
| (RMS No.) | | | |
| Leaf glossiness | Weak | Strong | Weak |
| Vein/midrib | Obliquely continuous | Obliquely continuous | Obliquely continuous |
| shape | throughout leaflet | throughout leaflet | throughout leaflet |
| Vein/midrib | 150d | 144c | 154d |
| color | | | |
| Aroma | Citrus zest with | Spicy | Earthy, but bitter |
| | chocolate and ginger | | |
| | undertones | | |

### III. Stem

| Charac- | | Parental variety(B3) | Parental variety(S9) |
|---|---|---|---|
| teristics | New Variety | (Female plant) | (Male plant) |
| Stem shape | Hollow, ribbed, | Hollow, ribbed, | Hollow, glandular, |
| | large | textured | ribbed |
| Stem | 2.50 cm | 2.8 cm | 1.9 cm |
| diameter | | | |
| at base | | | |
| Stem color | 139d | N144d | 195c |
| (RMS No.) | | | |

### IV. Inflorescence (Female/Pistillate Flowers)

| | | Parental variety(B3) | Parental variety(S9) |
|---|---|---|---|
| Characteristics | New Variety | (Female plant) | (Male plant) |
| Flowering | Elongated compound | Elongated compound | Elongated compound |
| (blooming) | spikes or panicles, from | spikes or panicles, from | spikes or panicles, from |
| habit | 0.50 m-1.20 m in length | 0.50 m-1.20 m in length | 0.50 m-1.20 m in length |
| Proportion of | 100% | 100% | 100% |
| female plants | | | |
| Inflorescence | Above | Even | Above |
| position | | | |
| Flower | Free | Overlapping-Touching | Free-Overlapping |
| arrangement | | | |
| Number of | 120-220 flowering sites | 350-550 flowering sites | 160-300 flowering sites |
| flowers per | | | |
| plant | | | |
| Flower shape | More or less sessile and | More or less sessile and | More or less sessile and |
| | are borne in racemes; | are borne in racemes; | are borne in racemes; |
| | calcaratre-urceolate; a | calcaratre-urceolate; a | calcaratre-urceolate; a |
| | small green bract | small green bract | small green bract |
| | enclosing the ovary with | enclosing the ovary with | enclosing the ovary with |
| | two long, slender stigmas | two long, slender stigmas | two long, slender stigmas |
| | projecting well above the | projecting well above the | projecting well above the |
| | bract | bract | bract |
| Flower | 0.50 cm | 0.70 cm | 1.00 cm |
| (individual | | | |
| pistillate) | | | |
| length | | | |
| Flower | 4.50 cm | 3.8 cm | 3.2 cm |
| (compound | | | |
| cyme) | | | |
| diameter | | | |
| Corolla | The inner envelope of | The inner envelope of | The inner envelope of |
| | floral leaves of a flower, | floral leaves of a flower, | floral leaves of a flower, |
| | of delicate texture and of | of delicate texture and of | of delicate texture and of |
| | some color other than | some color other than | some color other than |
| | green | green | green |
| Corolla Color | n/a | n/a | n/a |
| (RMS No.) | | | |
| Bract shape | Urceolate | Urceolate | Urceolate |
| Bract color | 142c | N134c | 143c |
| (RMS No.) | | | |
| Calyx shape | No defined calyx | No defined calyx | No defined calyx |
| Calyx color | n/a | n/a | n/a |
| (RMS No.) | | | |
| Stigma shape | Linear-lanceolate | Acute | Linear |
| Stigma length | 3.10 mm | 2.2 mm | 5.10 mm |
| Stigma color | 157c | 159d | 157d |
| (RMS No.) | | | |
| Trichome shape | Capitate-stalked | Capitate-stalked | Capitate-stalked |
| | glandular | glandular | glandular |
| Trichome color | 157a | 157a | 157a |
| (RMS No.) | | | |
| Terminal bud | Elliptical | Oblong | Elliptical |
| shape | | | |
| Terminal bud | 136b | 203c | 136d |
| color (RMS | | | |
| No.) | | | |
| Pedicel | Absent | Absent | Absent |
| Staminate shape | n/a | n/a | n/a |
| Pollen | Absent | Absent | Absent |
| description | | | |
| Seed | 2.10 to 2.80 mm | 1.80-2.30 mm, smooth | 2.80-3.30 mm, |
| description | textured and globular | and globular | globular |
| Petal | Apetalous | Apetalous | Apetalous |
| description | | | |
| Max THC | About 18.77~23.19% | About 18.88~19.37% | About 16.11~18.21% |
| content | | | |
| Max CBD | 0.00% | 0.00% | 0.00% |
| content | | | |
| Max CBG | About 0.98~1.78% | About 0.84~0.91% | About 0.67~0.95% |
| content | | | |

### V. Other Characteristics

| | | Parental | Parental |
|---|---|---|---|
| Charac- | | variety(B3) | variety(S9) |
| teristics | New Variety | (Female plant) | (Male plant) |
| Time | 9-11 weeks | 7-9 weeks | 9-11 weeks |
| period and | | | |
| condition of | | | |
| flowering/ | | | |
| blooming | | | |
| Hardiness | Hardy to | Hardy to | Hardy to |
| of plant | 25° F.—ambient | 25° F.—ambient | 25° F.—ambient |
| | temperature | temperature | temperature |
| Breaking | Flexible, resistant | Strong, non- | Flexible, resistant |
| action | to breakage | flexible | to breakage |
| Rooting rate | 99%—vigorous | 70%—moderate | 70%—moderate |
| after | | | |
| cutting/ | | | |
| cloning | | | |

The following is a detailed description of the new cultivar of ‘LEMON CRUSH ’. The following description is for plants that are 93-100 days old as of the time of the measurements.
General description:
   Plant life form and habit.—An herbaceous, upright, tap-rooted annual plant.
Classification:
   Denomination.—‘LEMON CRUSH’.
   Species.—Cannabis hybrid (mixed background).
Origin, form, and growth characteristics:
   Origin.—Progeny of the cross between BLK03 and SLV09.
   Propagation.—The strain is asexually propagated by cutting and cloning.
   Propagation ease.—Easy.
Plant:
   Height.—1.5-4 m.
   Width.—89 cm.
   Vigor.—High (very vigorous).
   Pest susceptibility.—Resistant.
   Time to harvest.—11 weeks.
   Genetically modified organism.—No.
Leaf/foliage:
   Structure.—Linear-lanceolate leaflet blades with glandular hairs.
   Shape.—Palmately compound.
   Arrangement.—Alternate.
   Margin.—Dentate, coarsely serrated, and the teeth point towards the tip.
   Hair.—Present.
   Leaf (with petiole) length at maturity.—19.1 cm.
   Number of leaflets.—3-9.
   Middle largest leaflet length.—13.4 cm.
   Middle largest leaflet width.—2.6 cm.
   Middle largest leaflet length/width ration.—13.4:2.6.
   Number of teeth of middle leaflet (average).—29.
   Color.—Upper side — 139a.
   Color.—Lower side — 139c.
   Leaf glossiness.—Weak.
   Veins/midrib shape.—Obliquely continuous throughout leaflet.
   Vein/midrib color.—150d.
Petiole:
   Petiole length.—5.5 cm.
   Petiole color.—149b.
   Stipule shape.—Acute-bulbous.
   Stipule length.—0.5 cm.
   Stipule color.—203b.
Stem:
   Shape.—Hollow, ribbed, and large.
   Diameter.—2.5 cm at base.
   Color.—139d.
Inflorescence:
   Blooming/flowering habit.—Elongated compound spikes or panicles, from 0.50 m-1.20 m in length.
   Inflorescence position relative to foliage.—Above.
   Flower arrangement.—Free.
   Number of flowers per plant.—120-220 flowering sites.
Flowers:
   Shape.—More or less sessile and are borne in racemes; calcaratre-urceolate; a small green bract enclosing the ovary with two long, slender stigmas projecting well above the bract.
   Flower (individual pistillate) length.—0.5 mm.
   Flower (compound cyme) diameter.—4.5 cm.
   Corolla shape.—The inner envelope of floral leaves of a flower, of delicate texture and of some color other than green.
   Corolla color.—N/A.
   Bract shape.—Urecolate.
   Bract color.—142c.
   Stigma shape.—Linear-lanceolate.
   Stigma length.—3.1 mm.
   Stigma color.—157c.
   Trichome shape.—Capitate-stalked glandular.
   Trichome color.—157a.
   Cola (terminal bud).—Elliptical.
   Cola (terminal bud) color.—136b.
   Pedicel.—Absent.
   Pedicel color.—N/A.
   Staminate flower.—N/A; no staminate flowers observed.
   Pollen.—N/A; no staminate flowers observed.
   Seed.—Textured and globular.
   Seed length.—2.1 to 2.8 mm.
   Petal.—Apetalous (without petals); This part is fused and appressed to the base of the ovary with the calyx and the perianth in the cannabis flowers.
Other characteristics:
   Aroma.—Citrus zest with chocolate and ginger undertones.
   Flowering/blooming period.—9-11 weeks.
   Hardiness.—Hardy to 25° F.-ambient temperature.
   Breaking action.—Flexible, resistant to breakage.
   Rooting rate after cutting/cloning.—99% vigorous.

1. A new and distinct cultivar of cannabis plant named ‘LEMON CRUSH’ substantially as shown and described herein.
